(19) United States
(12) Patent Application Publication
(10) Pub. No.: US 2005/0124701 A1
(43) Pub. Date: Jun. 9, 2005

(54) Method of targeting a therapeutic agent

(76) Inventors: Gregory T. Went, Mill Valley, CA (US); Jan WF Wasley, Guilford, CT (US); Stuart A. Lipton, Rancho Santa Fe, CA (US); James W. Larrick, Woodside, CA (US); Laurence R. Meyerson, San Rafael, CA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/868,044

(22) Filed: Jun. 14, 2004

Related U.S. Application Data
(60) Provisional application No. 60/477,511, filed on Jun. 11, 2003.

(51) Int. Cl. A61K 31/13

(52) U.S. Cl. 514/662

(57) ABSTRACT

Disclosed are conjugates in which an aminoadamantane derivative, such as amantadine, memantine, or rimantadine is linked to a therapeutic agent. The conjugate can then be used to target the therapeutic agent to an injured neuron.

### RELATED APPLICATIONS

This application claims priority to U.S. Ser. No. 60/477,511, filed Jun. 11, 2003. The contents of this application are incorporated herein by reference in their entirety.

### FIELD OF THE INVENTION

The invention relates generally to methods of targeting therapeutic agents to neurons.

### BACKGROUND OF THE INVENTION

Acute and chronic neurological and neuropsychiatric diseases are among the leading causes of death, disability, and economic expense in the world. One of the main challenges in developing treatments for these diseases is the difficulty in getting therapeutic agents across the blood-brain barrier.

Certain uncompetitive NMDAR antagonists, such as memantine, readily cross the blood-brain barrier, achieving nearly identical concentrations in the extra-cellulary fluid surrounding brain tissue and systemic serum. In addition, these antagonists are believed to work by blocking the excessive activation of N-methyl-D-aspartate-type glutamate receptors (NMDAR) in the brain, there by reducing excessive Ca²⁺ influx through the receptor's associated ion channel. Glutamate excitotoxicity has been implicated in neuronal injury and death due to either necrosis or apoptosis.

### SUMMARY OF THE INVENTION

The invention is based in part on the discovery that memantine and related aminoadamantanes, which are uncompetitive inhibitors of NMDAR that block the receptor in excitotoxic conditions characteristic of damaged neurons, can be used to selectively target therapeutic agents to the brain, and more specifically to excitotoxic neurons. Accordingly, the invention provides conjugates in which an aminoadamantane derivative, such as amantadine, memantine, or rimantadine is linked to a therapeutic agent. The conjugate can then be used to target the therapeutic agent to an injured neuron.

In one aspect, the invention features a method of targeting a therapeutic agent to the brain by administering to a subject in need thereof a conjugate that includes an aminoadamantane derivative linked to the therapeutic agent. In some embodiments, the method further includes identifying a therapeutic agent in which targeting to the brain of a subject is desired.

In some embodiments, the therapeutic agent delivered in the conjugate reaches a Tmax at from between about 0.1 hours to about 5 hours after administration to the subject, e.g., the Tmax at from about 0.25 hours to about 2 hours, or about 0.5 hours to about 1 hour.

In some embodiments, the Tmax is determined by determining the levels in the brain or cerebrospinal fluid of a subject. In some embodiments, the Tmax of the therapeutic agent in the brain is at least 4-fold that of the Tmax of the therapeutic agent administered when not conjugated to the aminoadamantane derivative, e.g., the Tmax can be at least 2-fold that of the Tmax obtained when the therapeutic agent is administered not conjugated to the aminoadamantane derivative or at least 1.5 fold that of the Tmax of the therapeutic agent administered when not conjugated to the aminoadamantane derivative.

In some embodiments, the Tmax is determined by determining the levels in serum of a subject.

In some embodiments, levels of the conjugate are determined by measuring the Cmax of the conjugate. In some embodiments, the Cmax is determined by determining the levels in the brain- or cerebrospinal fluid of a subject.

In various embodiments, the therapeutic agent delivered as part of the conjugate reaches Cmax in brain in no more than about 0.25, 0.5, or 1, 2, 4, 6, 8, 16, or 24 hours after administration of the conjugate, e.g., the conjugate reaches Cmax in the brain in no more than about 1 hour after administration of the conjugate.

In various embodiments, at the time Tmax, the ratio of the brain concentration Cmax to the corresponding concentration in serum is 1.5-fold, 1.75-fold, 2-fold, 3-fold, 4-fold, or more of that attained by the unconjugated therapeutic agent, e.g., in some embodiments the ratio of the brain concentration Cmax to the corresponding concentration in serum at that time is 4-fold that attained by the unconjugated therapeutic agent or the ratio of the brain concentration Cmax to the corresponding concentration in serum at that time is 4-fold that attained by the unconjugated therapeutic agent (when not conjugated to the aminoadamantane derivative).

In various embodiments, the T½ (half life) in the brain of the conjugated therapeutic agent is at least 2-fold that of the T½ in the brain of the unconjugated therapeutic agent, e.g., the T½ of the conjugated therapeutic agent is at least 4-fold that of the unconjugated therapeutic agent or at least 8-fold of the T½ of the unconjugated therapeutic agent.

In some embodiments, the Cmax is determined by determining the levels in serum of a subject.

In some embodiments, the therapeutic agent is a neuroprotective agent.

In some embodiments, the therapeutic agent is for treating a disorder associated with excessive NMDAR activity.

In some embodiments, the therapeutic agent is non-nitrosylated or is a non-NO-generating therapeutic agent.

In some embodiments, the therapeutic agent is for treating a neurological disorder (e.g., a neurological disease, condition or syndrome). The neurological disorder is, e.g., stroke, other forms of hypoxic injury, haemorrhagic brain injury, traumatic brain injury, spinal cord injury, familial Alzheimer's disease (FAD), Parkinson's disease, ALS (amyotrophic lateral sclerosis), neuroprotection in epilepsy, a metabolic disorder, hypoglycemia, encephalopathy, tumors and malignancies (brain, spinal cord, and systemic), cerebellar degenerations, and ataxias, migraine, vertigo, tinnitus and cochlear disorders, bowel syndromes, peripheral neuropathy, metabolic bone disease and osteoporosis, obesity, and diabetes and pre-diabetic syndromes, glaucoma, HIV associated dementia neuropathic pain, Huntington's disease or other dementing disease, anxiety, depression or withdrawal from drug (or opiate) addiction or drug (or opiate) dependency, minimal cognitive impairment (MCI), Down's syndrome, normal cognitive senescence, meningitis, sepsis and septic encephalopathy, CNS vasculitis, schizophrenia, alcoholic diseases, multiple sclerosis or other demyelinating disease, leukodystrophies and X-ADL, childbirth and surgical anesthesia. Treatment also provides neuroprotection from cerebrovascular risk factors and post-ischemic neurovascular syndromes.

In some embodiments, the therapeutic agent is for treating hyperhomocysteinemia contributing to atheroslcerotic and other degenerative disease processes.

In some embodiments, the therapeutic agent is for treating head trauma or spinal cord injury.

In some embodiments, the therapeutic agent is for treating demyelinating disease, which can include multiple sclerosis.

In some embodiments, the therapeutic agent is a caspase inhibitor, a superoxide dismutase mimetic, calcium chelator, MAPK antagonist, an ERK-MAPK antagonist, a p38 MAPK inhibitor, a cytochrome C chelating antibody, APAF-1 inhibitor, AIF (apoptosis-inducing factor [caspase-independent]) inhibitor/Apoptosis inhibitor, gamma vinyl GABA (GVG, vigabatrin), PARP inhibitor, an NOS inhibitor, a dopamine agonist, a dopamine analog, an immunosuppressant, an anti-inflammatory, an anti-cancer agent, a statin, an anti-epilepsy agent, a cannabinoid, an anti-viral, a nootropic (cognitive enhancer), an M-2 agonist, an obesity treatment agent, or a non-steroidal anti-inflammatory drug (NSAID).

In some embodiments, the adamantine derivative is neuroprotective. Preferably, the aminoadamantane derivative binds to an N-methyl-D-aspartate (NMDA) receptor expressed on an injured neuron.

Examples of suitable aminoadamantane derivatives include, e.g., memantine, rimantadine (1-(1-aminoethyl)adamantane), and amantadine (1-aminoadamantane)

In some embodiments, the therapeutic agent is attached to the aminoadamantane derivative in the conjugate at the 1-(bridgehead) position or the 2-position.

In some embodiments the therapeutic agent is attached to the aminoadamantane derivative via a metabolically cleavable linkage.

In another aspect, the invention provides a method of targeting a therapeutic agent to an injured neuron of a subject by administering to a subject in need thereof a conjugate that includes an aminoadamantane derivative linked to the therapeutic agent. In some embodiments, the injured neuron is in the brain of the subject. The injured neuron can be, e.g., in the central nervous system (CNS).

In a still further aspect, the invention provides a method of lowering the neurotoxicity of a therapeutic agent in a subject by administering to the subject a conjugate that includes an aminoadamantane derivative linked to said therapeutic agent.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is an example of a synthesis for a conjugate of an aminoadamantane derivative and a therapeutic agent.

FIG. 2 is an example of a synthesis for a conjugate of an aminoadamantane derivative and a therapeutic agent.

FIG. 3 is an example of a synthesis for a conjugate of an aminoadamantane derivative and a therapeutic agent.

FIG. 4 is an example of a synthesis for a conjugate of an aminoadamantane derivative and a therapeutic agent.

FIG. 5 is an example of a synthesis for a conjugate of an aminoadamantane derivative and a therapeutic agent.

### DETAILED DESCRIPTION OF THE INVENTION

The invention provides compositions and methods useful for acute, chronic and/or prophylactic treatment of neurologic and neurodegenerative diseases. The compositions and methods are also useful for attenuating acute or chronic neuronal damage in neurological disease (“neuroprotection”), and prophylaxis of neurological diseases.

For example, the compositions and methods are useful for treating neurological diseases that involve excessive stimulation of the NMDA receptor, hypofunction of the NMDA receptor, up- or down regulation of the NMDA receptor, or abnormal subunit structure or function of the NMDA receptor.

Neuroprotective efficiency is achieved by using the aminoadamantane derivative to specifically deliver the therapeutic agent (e.g., a neuroprotective agent) to the brain or injured neuron. The aminoadamantane derivative in the conjugates allows for the therapeutic agent to be delivered in a lower systemic or topical dose than that which would be required if the therapeutic agent were administered alone. The lower dose also minimizes the side effects and/or toxic effects that may be observed when the therapeutic agent is administered alone and thus is more likely to interact undesirably with healthy neurons and other healthy tissues.

The conjugates described herein thus provide a way to achieve effective drug concentrations at physiologically protected sites (e.g., in the brain) and a way to reach therapeutically-effective levels after systemic administration of much lower levels than are currently administered to achieve a therapeutic dose of the conjugated therapeutic agent. Administration of the therapeutic agent as part of the conjugate additionally results in results in decreased systemic metabolism, degradation and toxicity, reduced systemic adverse drug interactions, and generally reduced side effects. These biological effects can also be obtained with simplified dosage schedules, particularly for drugs with short systemic half-lives.

Therapeutic Agents

Therapeutic agents that are linked to the aminoadamantane compounds (e.g., amantadine, memantine or rimantadine) to form the conjugates of the invention include, e.g., anticonvulsive agents, antiparkinsonian drugs, caspase inhibitors, superoxide dismutase mimetics, calcium chelators, calcium channel blockers, gamma-aminobutyric acid (GABA) receptor agonists, antagonists, and uptake inhibitors and enhancers, p38 mitogen-activated protein kinase (MAPK) antagonists, ERK-MAPK antagonists, cytochrome C chelating antibodies; APAF-1 (apoptotic protease activating factor-1) inhibitors, AIFs (apoptosis inhibiting factors), PARP (poly (ADP-ribose) polymerase) inhibitors, anti-epileptic agents, immunosuppressants, anti-inflammatory agents, non-steroidal anti-inflammatory drugs (NSAIDs), anti-cancer agents, statins, cannabinoids, anti-virals, nootropic (cognitive enhancer) agents, alkaloids, catecholamines including dopamine analogues and derivatives, muscarinic receptor agonists and antagonists, cholinergic receptor agonists and antagonists, obesity treatment agents, and NOS (nitric oxide synthetase) inhibitors, phenothiazines, thioxanthemes and related compounds; clozapine, haldoperidol, loxapine, benzodiazapene antidepressants of the norepinephrine reuptake inhibitor type; monoamine oxidase inhibitors; antidepressants and antimanic agents, antioxidants and other compounds that mitigate the effects of reactive oxygen species (for the treatment of Alzheimer's disease, Parkinson's disease, or other neurodegenerative conditions such as ataxia telangiectasia and amyelolaterosclerosis (ALS)).

In some embodiments, the therapeutic agent is non-nitrosylated or is a non NO-generating therapeutic agent.

Dopamine agonists suitable for use in the conjugates include, e.g., the aminotetralins; treatments for damage caused by stroke include conjugates of immunosuppressants such as tacrolimus (FK-506); treatments for brain inflammation include conjugates of dexamethasone; treatments for brain cancer include conjugates of methotrexate, vinca alkaloids, carmustine, cisplatin, nitrosourea, hydroxyurea, and procarbazine; treatments for brain inflammation secondary to beta amyloid plaque formation include conjugates of statins; neuroprotectant conjugates include cannibinoids (which increase appetite and reduce agitation in a subject; conjugates useful for treating viral encephalitis include acyclovir; conjugates useful for treating obesity include CCK fragments; conjugates useful for treating ALS include riluzole; and conjugates useful for treating neuroleptic maignant syndrome include methylprednisolone; conjugates useful for treating schizophrenia include dopamine antagonists, including those agents which interact with the D1 or D5 receptor subtypes.

Suitable anti-epileptic agents that can be used in the conjugates include, e.g,. sodium channel inhibitors, for example, phenytoin, carbamazepine, oxcarbazepine; GABA receptor modulators, for example, phenobarbital, tiagabine, vigabatrin (γ-vinyl GABA, a GABA transaminase inhibitor), gabapentin; agents that reduce calcium currents, T currents, for example, ethosuccimide and zonisamide; and those that exert unknown or multiple effects, for example, piracetam, levetiracetam, aniracetam, nefiracetam or topiramate.

Suitable nootropic agents include sedrafinil, centrophenoxine, deprenyl, dehydroepiandrosterone (DHEA), dimethylaminoethanol (DMAE), Gingko Biloba, piracetam, pyroglutamate, Vinpocetine, and xanthinol nicotinate.

Other therapeutic agents include NSAIDs such as diclofenac, piroxicam (Feldene), and indomethacin, acetaminophen, ibuprofen, naproxen and ketoprofen, including nitrosylated analogs thereof; COX-2 (cyclooxygenase) inhibitors, including rofecoxib (VIOXX®, or 4-[4-(methylsulfonyl)phenyl]-3-phenyl-2(5H)-furanone), celecoxib (CELEBREX®, or 4-[5-(4-methylphenyl)-3-(trifluoromethyl)-1H-pyrazol-1-yl]benzenesulfonamide), and valdecoxib (BEXTRA®, or 4-(5-methyl-3-phenyl-4-isoxazolyl) benzenesulfonamide), and statins such as lovastatin, simvastatin, pravastatin, fluvastatin, and atorvastatin; and NOS inhibitors include arginine and citrulline analogs such as L-Thiocitrulline, S-alkyl-L-thiocitrullines (e.g., S-Methyl-L-thiocitrulline dihydrochloride), NG-Nitro-L-arginine methyl ester (L-NAME), 7-Nitroindazole monosodium salt (7-NINA), 7-Nitroindazole and related substituted indazoles, NG-Monomethyl-L-arginine acetate (L-NMMA), NG-N-nitro-L-arginine (L-NNA), 1-(2-Trifluoromethylphenyl) imidazole (TRIM), L-NIO (an L-ornithine analog which has an iminoethyl group instead of an amine group), L-NIL (an L-lysine analog which as an iminoethyl group instead of an amine group), Diphenyleneiodonium chloride, 3-Br-7-Nitroindazole, isothiourea derivatives (such as S-Methyl-ITU, S-Ethyl-ITU, S-Isopropyl-ITU, and S-Aminoethyl-ITU), 2-Iminopiperidine, and DHAP.

The therapeutic agent in the conjugates of the invention can additionally include an agonist of a muscarinic receptor. Such agonists include Alvameline, Arecoline, Cevimeline, PD-151832, Milameline, CI-979A, RU35926, Sabcomeline, SB 202026, SCH-217443, SDZ-210-086, SR 46559A, Talsaclidine, WAL 2014-FU, Tazomeline, Xanomeline, and YM 796.

Some representative examples of therapeutic agents include the following (* indicates a potential site of conjugation via an appropriate linker): the caspase inhibitor CGP 82630 (IDN-7866) from IDUN/Novartis: 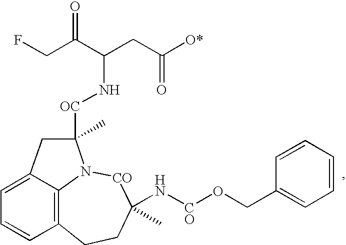
the superoxide dismutase stimulator SC 55858 from Pharmacia 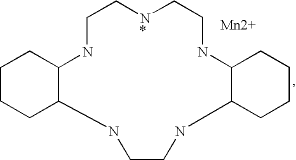
the p38 MAP kinase inhibitor from Bayer: 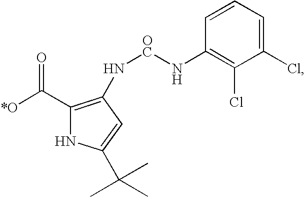
the calcium chelator DP-b-99 from D-Pharm, Ltd.: 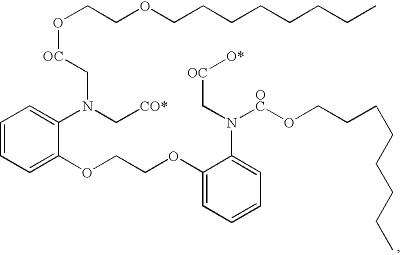
the ERK-MAP kinase inhibitor CEP 1347 from Cephalon, Inc: 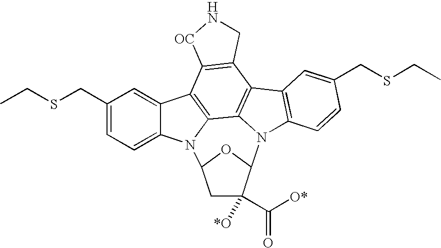
the apoptosis inhibitor VX-799 from Vertex: 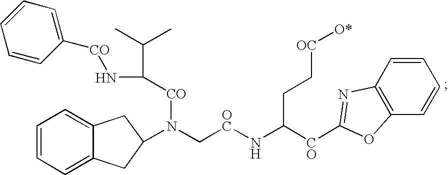
the PARP1 inhibitor FR 247304, from Fujisawa: 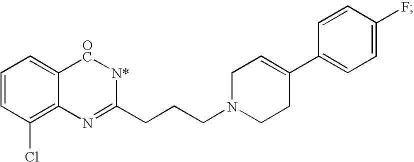
the NO Synthase inhibitor (Schering AG): 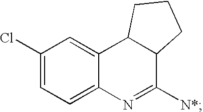
the tricyclic antidepressant MK-801 from Sigma: 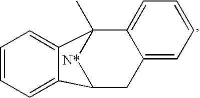
the anti-epileptic agent vigabatrin: 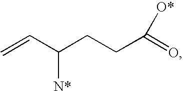
the anti-epileptic agent piracetam: 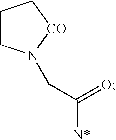
the anti-epileptic agent levetiracetam: 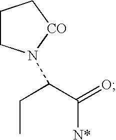
the anti-epileptic agent nefiracetem: 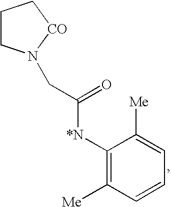
and the anti-epileptic agent topiramate:

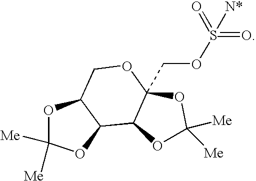

Other therapeutic agents include serofendic acid, PBAS ((5-pentafluorobenzyl) aminosalicylic acid), cannibinoids, such as CB-1 antagonists (Sanofi), and CB-2 agonists (Mak Scientific), and anadamide.

Aminoadamantane Derivatives

Preferably, the aminoadamantane derivative is easily measured in subject's body, e.g., by measuring serum levels of the aminoadamantane derivative available. The aminoadamantane derivative is in addition preferably well-tolerated in the subject and has minimal side effects.

Preferably, the aminoadamantane derivative itself has desirable therapeutic properties, e.g., the aminoadamantane derivative demonstrates anticonvulsant, neuroprotective properties and/or dopaminergic effects.

In some embodiments, the aminoadamantane derivative quickly achieves maximal CNS concentrations within hours, and lingers for 24-72 hours in the CNS.

One suitable aminoadamantane derivative that can be present in the conjugate is memantine (1-amino-3,5-dimethyladamantane of the adamantane class). A preferred adamantine derivative has no active metabolites that possess NMDA antagonizing properties. Other preferred aminoadamantane derivatives that can be used include those that have been approved for therapeutic use in humans, e.g., rimantadine (1-(1-aminoethyl)adamantane), and Amantadine (1-aminoadamantane).

Linking Aminoadamantane Derivatives to Therapeutic Agents

The therapeutic agent can be linked to the aminoadamantane derivative at any suitable position on the admantane derivative. The aminoadamantane derivative is optionally attached via a linker.

The linkage can be metabolically stable (i.e., the therapeutic agent remains attached to the aminoadamantane compound) or metabolically or physiologically labile, i.e., unstable, wherein the therapeutic agent is released from the aminoadamantane compound.

A linker that is labile under certain conditions can be selected (e.g., at a certain pH)). Metabolically cleavable linkages are not equally labile or ‘cleavable’, particularly in terms of their rates of cleavage. The ease of metabolic cleavage has the following rank order: —COOR>CONH>CH═N>SO₂NH>CH₂N, i.e., the ester is the most readily cleaved and the alkylamine is the most stable. Even in the case of the esters, the rate of cleavage can be controlled by further substitution on the alpha (adjacent) carbon atom. In general, a more extensive amount of substitution makes cleavage more difficult, i.e., makes for a more stable linkage.

For example, the type of linker used in the conjugate to attach the therapeutic agent can facilitate hydrolytic release of the therapeutic agent at an intracellular site. In other embodiments, the type of linker used in the conjugate to attach the therapeutic agent facilitates the enzymatic release of the therapeutic agent at a target site. In some embodiments, the linker functional group is hydrolyzed by an enzymatic activity found in brain tissue, including neuronal, glial and other brain cell types, such as an esterase, including an esterase having a differential expression and activity profile in the appropriate target cell type. In additional embodiments, specific release of the therapeutic agent is achieved by enzymatic or chemical release of the therapeutic agent by extracellular cleavage of a cleavable linker moiety via an enzymatic activity specific for brain tissue, with resulting specific uptake of the released psychotropic, neurotropic or neurological agent by the appropriate cell in said tissue.

In some embodiments, each of the first and second functional linker groups are those which react with a hydroxyl group, a primary or secondary amino group, a phosphate group or substituted derivatives thereof, or a carboxylic acid group on the therapeutic agent or aminoadamantane derivative.

When the aminoadamantane derivative includes a primary, bridgehead amine, or the aminoadamantane compound (amantadine, memantine or rimantadine), the aminoadamantane derivative can be converted to the hydroxyl- or diamino-derivative, which may then conjugated to the therapeutic agent via a linker:

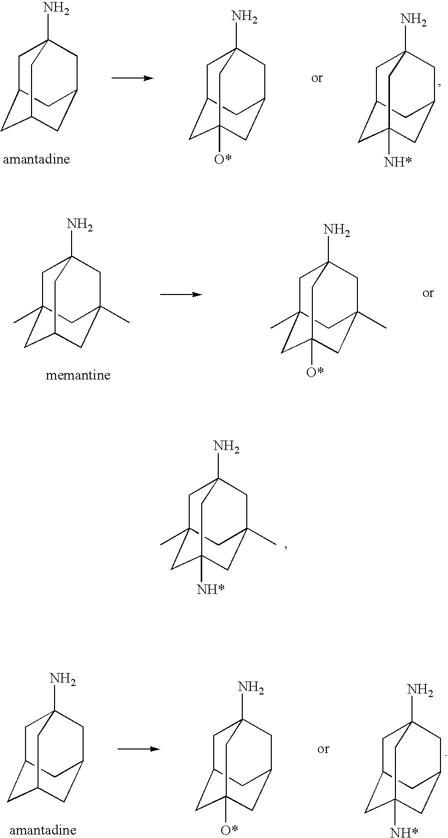

In some embodiments, the original, bridgehead amino group is conjugated to the linker.

Cleavable linkers include those that form an ester (COOR), amide (CONHR), sulfonamide (NHSO₂), sulfonate (SO₂R), or ether (ROR) with the corresponding functionality on the aminoadamantane and on the therapeutic agent. In some examples, the linker is an alkyl linker, in others, the linker is a heteroalkyl linker, in others two adjacent atoms in the linker are joined together to form a cycloalkyl, heterocyclyl, aryl, or heteroaryl group. Some linkers include the following, where n is 0-7:

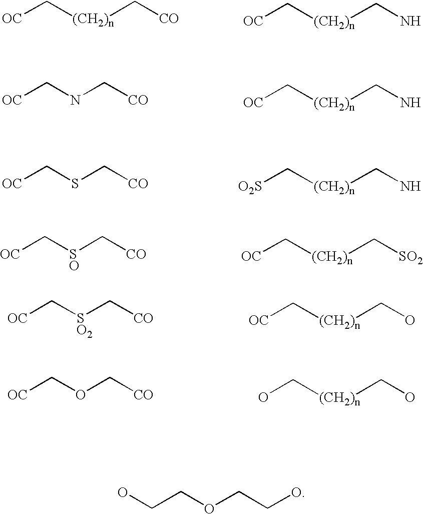

Other commercially available suitable linkers include the following:

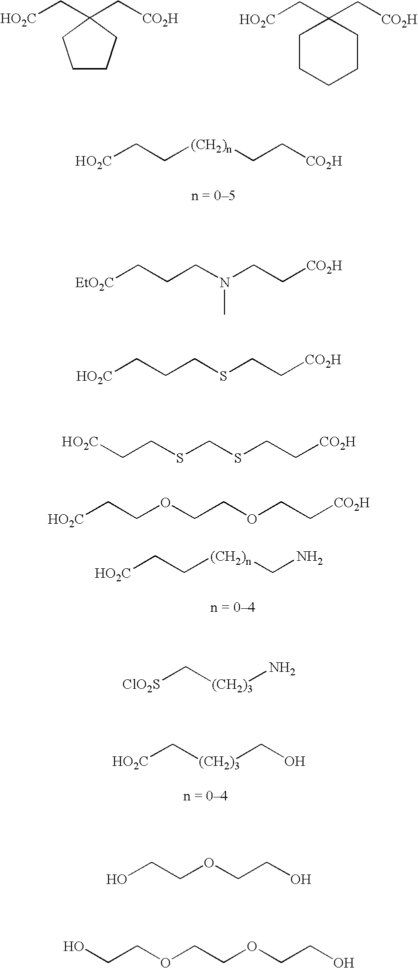

The terminal carboxyl functionalities of the linkers can be part of an acid chloride, ROC(O)Cl, (or reactive equivalent), which form a carbamate, ROC(O)NR, when reacted with an amine, the SO₂ reactants are sulfonyl chlorides, SO₂Cl, compounds (or reactive equivalent), which form a sulfonamide when reacted with an amine. Amines and ethers are formed from the reaction of a compound having an active leaving group: 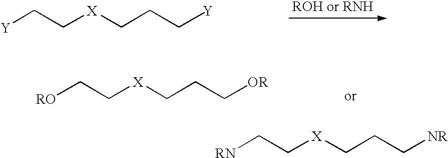
where Y is I, Br, mesyl, tosyl, etc and X is CH₂, O, S, NH, NR, SO, SO₂.

It may be necessary to modify a therapeutic agent so that it is reactive with a linker functional group to form a covalent bond. For example, the anti-epileptic agent aniracetam is modified such that the methoxy group (“OMe”) is hydrolyzed to form a reactive hydroxyl group (OH).

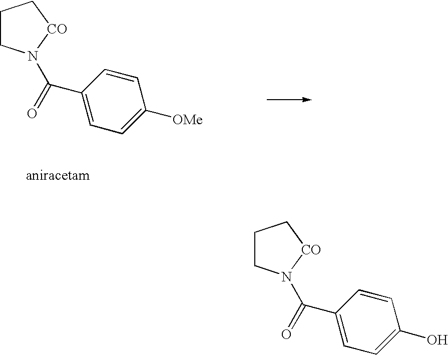

Thus, the aniracetam used for conjugation to an aminoadamantane is represented as: 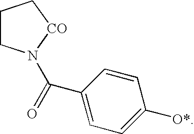
Examples of Conjugates

An illustration of various linkers used to prepare a conjugate from memantine (through the original, bridgehead amine) and the Bayer p38 MAP kinase inhibitor (through the carboxylic acid moiety) is presented in Scheme 1.

Scheme 1

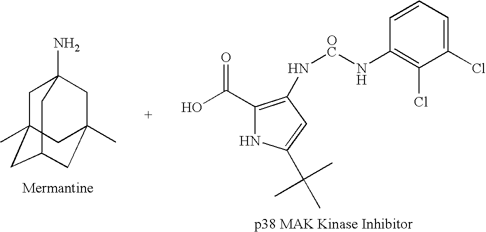

| Conjugate | Linker Used |
|---|---|
| 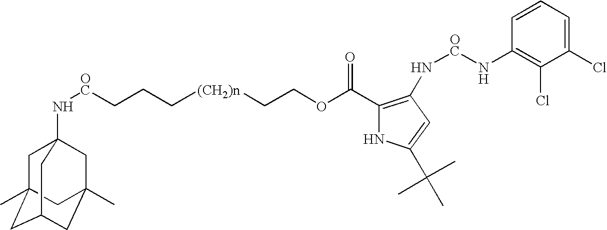 | 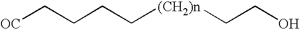 |
| 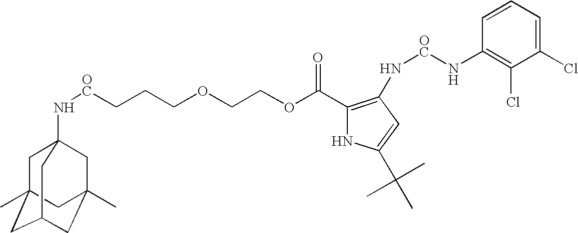 | 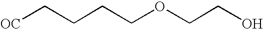 |
| 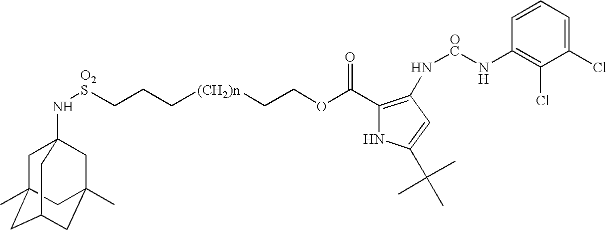 | 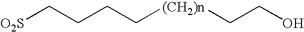 |
| 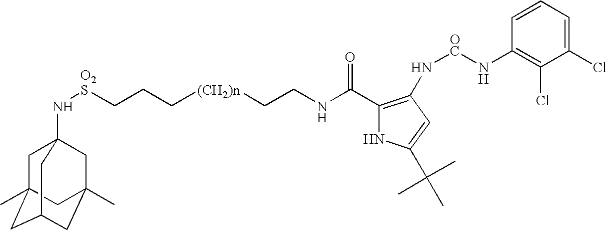 | 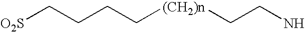 |
| 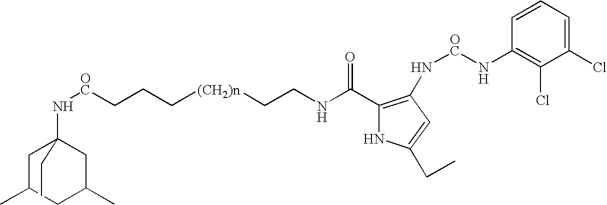 | 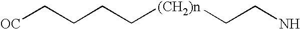 |

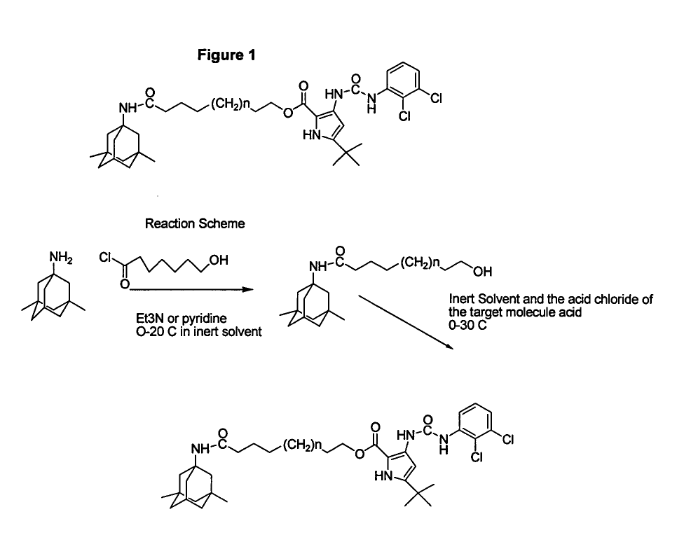

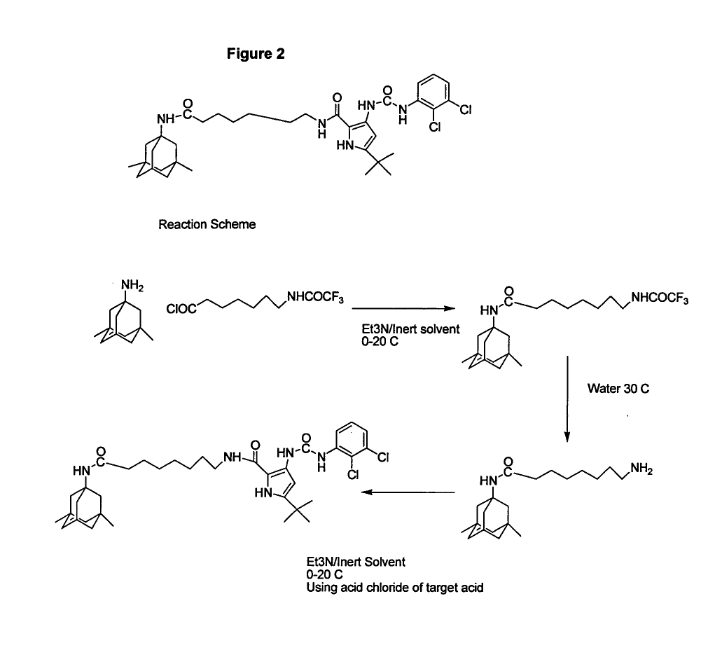

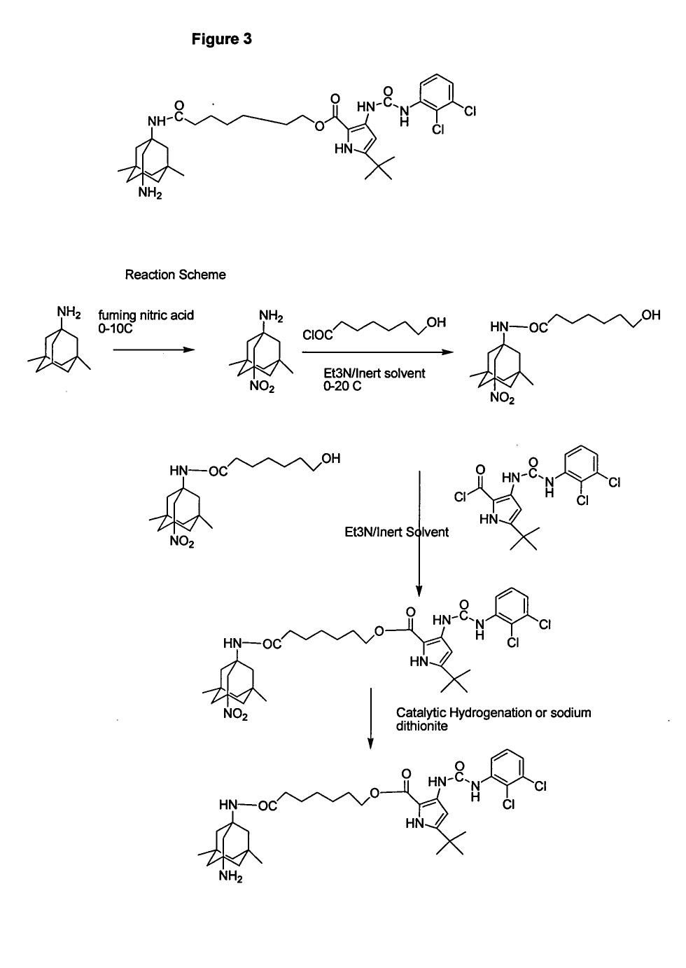

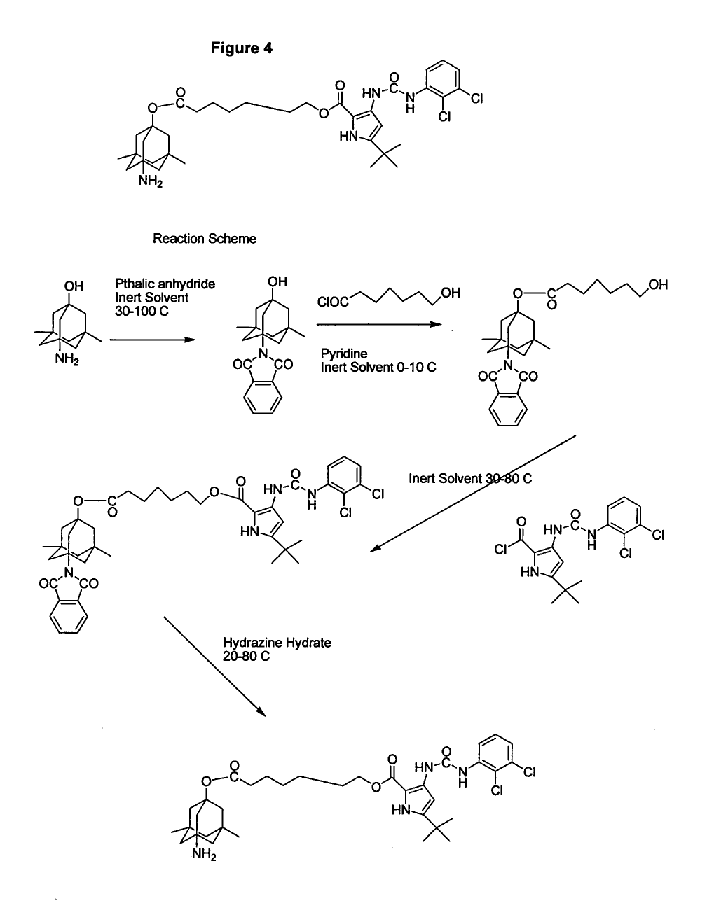

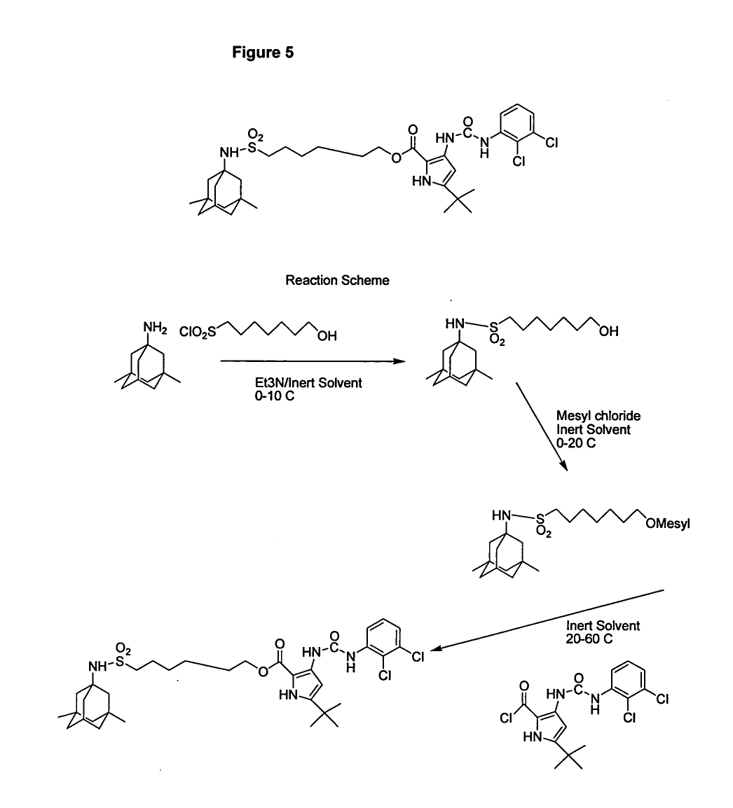

Examples of syntheses for these conjugates are presented in FIGS. 1-5.

In some conjugates, a therapeutic agent having a carboxylic acid functionality (COOH) is conjugated directly to the aminoadamantane amine via an amide bond, as shown below for memantine:

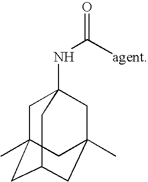

In some conjugates, the linker is a peptide, linking an amine from the aminoadamantane with a carboxylic acid on the therapeutic agent.

The spacer can optionally be a peptide of formula (amino acid)ₙ. In some embodiments, n is an integer between 2 and 25, preferably between 2 and 5. A memantine-tripeptide-therapeutic agent is shown below: 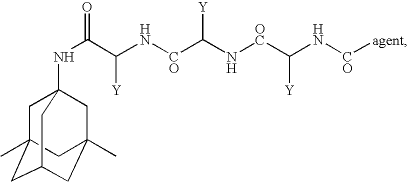
where “agent” represents the remainder of the therapeutic agent, and each Y represents any naturally occurring amino acid side chain.

Stable linkers include carbon linkages, and can be formed by reaction of the amino functionality on the aminoadamantane (or on the therapeutic agent) with a linker containing an aldehyde or ketone, to form a Schiff base, which is reduced to an aminoalkyl derivative. Synthesis of metabolically stable linkages is shown in Scheme 2.

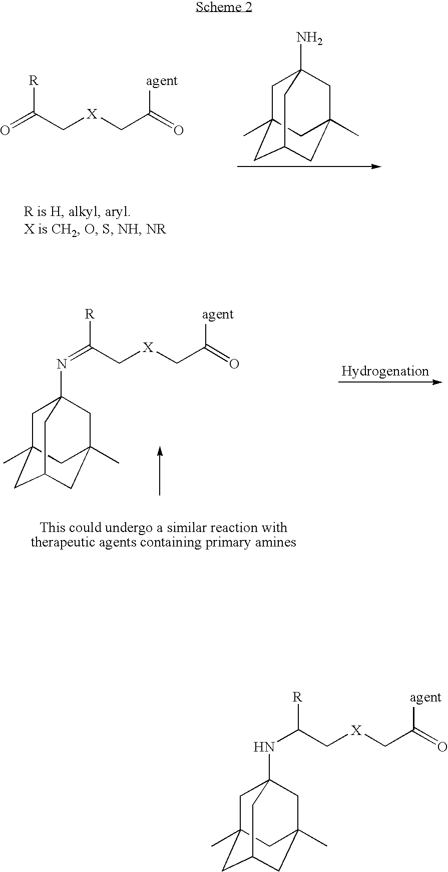

Ureas and thioureas are also useful linkages. Ureas and thioureas can be formed from a primary amine on the aminoadamantane and a primary amine on a therapeutic agent using the linkers depicted below:

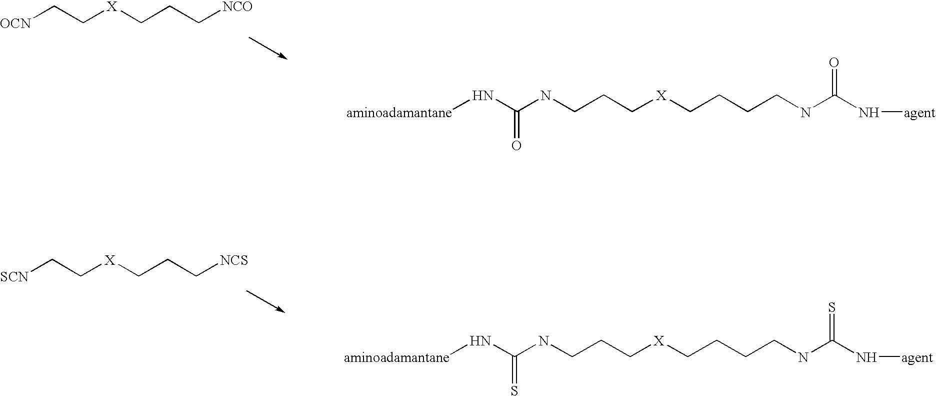

In some conjugates, the linker is attached to the aminoadamantane via a metabolically stable linker and attached to the therapeutic agent via a labile linkage (e.g., an ester or carboxamide) shown for a memantine-linker-therapeutic agent below: 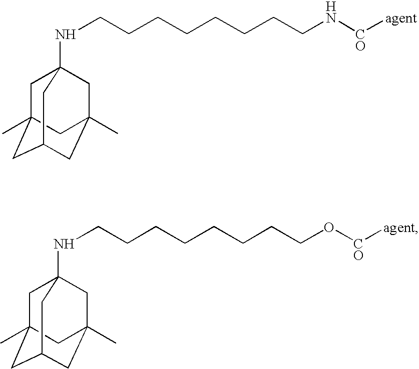
where “agent” refers to the remainder of the therapeutic agent which contained a carboxylic acid. A reaction scheme for the generation of the ester is shown in Scheme 3:

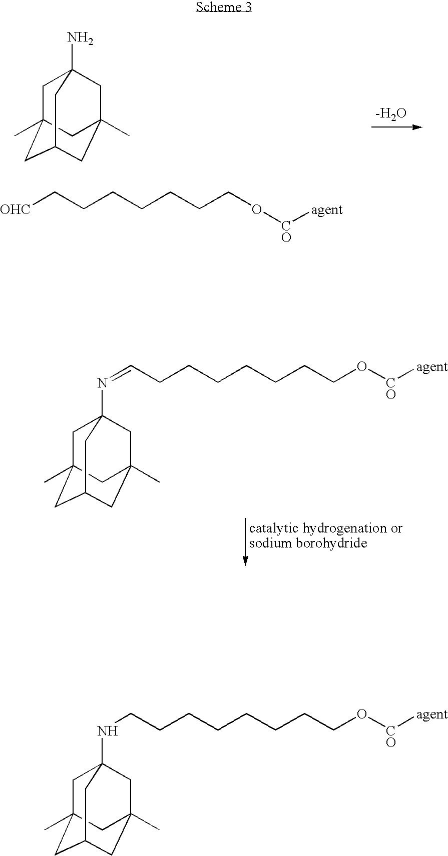

A similar scheme can be used to produce the carboxamide rather than the ester

Method of Using the Conjugates and Pharmaceutical Compositions Containing Conjugates

The conjugates are useful for treating a neurological condition or disorder, which can include a, e.g., neurological disease, condition or syndrome. The neurological disorder can also be, e.g., stroke, other forms of hypoxic injury, haemorrhagic brain injury, traumatic brain injury, spinal cord injury, mild cognitive impairment (MCI), Alzheimer's disease, e.g., familial Alzheimer's disease (FAD), Parkinson's disease, ALS (amyotrophic lateral sclerosis), epilepsy, a metabolic disorder, hypoglycemia, encephalopathy, tumors and malignancies (brain, spinal cord, and systemic), cerebellar degenerations, and ataxias, migraine, vertigo, tinnitus and cochlear disorders, peripheral neuropathy, obesity, and diabetes and pre-diabetic syndromes, glaucoma, HIV-associated dementia or other dementing disease, neuropathic pain, Huntington's disease, anxiety, depression or withdrawal from drug (e.g., opiate) addiction or drug (or opiate) dependency, Down's syndrome, normal cognitive senescence, meningitis, sepsis and septic encephalopathy, CNS vasculitis, schizophrenia, alcoholic diseases, multiple sclerosis or other demyelinating disease, leukodystrophies and X-ADL, nociceptive pain, childbirth and surgical anesthesia.

The conjugation can be also be used to provide neuroprotection in subjects with elevated cerebrovascular risk factors, and/or with and post-ischemic neurovascular syndromes. The conjugates are additionally useful for treating hyperhomocysteinemia contributing to atheroslcerotic and other degenerative disease processes.

The conjugates are typically administered to a patient in the form of a pharmaceutically acceptable salt or in a pharmaceutical composition. A compound that is administered in a pharmaceutical composition is mixed with a suitable carrier or excipient such that a therapeutically effective amount is present in the composition. The term “therapeutically effective amount” refers to an amount of the conjugate that is necessary to achieve a desired endpoint (e.g., decreasing neuronal damage).

In some embodiments, the compositions are suitable for internal use and include an effective amount of a pharmacologically active conjugate of the invention, alone or in combination, with one or more pharmaceutically acceptable carriers.

A suitable subject can be, e.g., a human, a non-human primate (including a gorilla or chimpanzee, or orangutan), a rodent (including a mouse, rat, guinea pig, or gerbil) a dog, a cat, horse, cow, pig, sheep, rabbit, or goat.

The conjugates are administered in amounts which will be sufficient to exert their desired biological activity. A variety of preparations can be used to formulate pharmaceutical compositions containing the conjugates, including solid, semi solid, liquid and gaseous forms. Remington's Pharmaceutical Sciences, Mack Publishing Company (1995) Philadelphia, Pa., 19th ed. Tablets, capsules, pills, powders, granules, dragees, gels, slurries, ointments, solutions suppositories, injections, inhalants and aerosols are examples of such formulations. The formulations can be administered in either a local or systemic manner or in a depot or sustained release fashion. Administration of the composition can be performed in a variety of ways. Among others, oral, buccal, rectal, parenteral, intraperitoneal, intradermal, transdermal and intratracheal means can be used.

Where the conjugate is given by injection, it can be formulated by dissolving, suspending or emulsifying it in an aqueous or nonaqueous solvent. Vegetable or similar oils, synthetic aliphatic acid glycerides, esters of higher aliphatic acids and propylene glycol are examples of nonaqueous solvents. The conjugate is preferably formulated in aqueous solutions such as Hank's solution, Ringer's solution or physiological saline buffer.

Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, preferably about 1 to 50%, of the active ingredient.

Liquid, particularly injectable compositions can, for example, be prepared by dissolving, dispersing, etc. The conjugate is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated. Injectable compositions are preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

The conjugates can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parental injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference. Where the conjugate is given orally, it can be formulated through combination with pharmaceutically acceptable carriers that are well known in the art. The carriers enable the compound to be formulated, for example, as a tablet, pill, suspension, liquid or gel for oral ingestion by the patient. Oral use formulations can be obtained in a variety of ways, including mixing the compound with a solid excipient, optionally grinding the resulting mixture, adding suitable auxiliaries and processing the granule mixture.

The conjugates of the invention can also be administered in such oral dosage forms as timed release and sustained release tablets or capsules, pills, powders, granules, elixirs, tinctures, suspensions, syrups and emulsions.

For instance, for oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine. Suitable excipients include sugars such as lactose, sucrose, mannitol or sorbitol; cellulose preparations such as maize starch, wheat starch, potato starch, gelatin, gum tragacanth, methyl cellulose, hydroxypropylmethylcellulose, sodium carboxymethylcellulose and polyvinylpyrrolidone (PVP).

Alternatively, the conjugates are delivered in an aerosol spray preparation from a pressurized pack, a nebulizer or from a dry powder inhaler. Suitable propellants that are used in a nebulizer include, for example, dichlorodifluoro-methane, trichlorofluoromethane, dichlorotetrafluoroethane and carbon dioxide. The dosage is determined by providing a valve to deliver a regulated amount of the compound in the case of a pressurized aerosol.

Furthermore, preferred conjugates for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.01% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like may be used. The conjugate defined above, may be also formulated as suppositories using for example, polyalkylene glycols, for example, propylene glycol, as the carrier. In some embodiments, suppositories are advantageously prepared from fatty emulsions or suspensions.

The conjugates of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the conjugates described herein can be provided as a complex with a lipophilic compound or non-immunogenic, high molecular weight compound constructed using methods known in the art. An example of nucleic-acid associated complexes is provided in U.S. Pat. No. 6,011,020.

The conjugates of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the conjugates of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, triethanolamine oleate, etc.

The dosage regimen utilizing the conjugates is selected in accordance with a variety of factors including type, species, age, weight, sex and medical condition of the patient; the severity of the condition to be treated; the route of administration; the renal and hepatic function of the patient; and the particular conjugate employed. An ordinarily skilled physician or veterinarian can readily determine and prescribe the effective amount of the drug required to prevent, counter or arrest the progress of the condition.

Pharmaceutical compositions typically contain a therapeutically effective amount of the conjugate. The amount of the conjugate will depend on the patient being treated. The patient's weight, severity of illness, manner of administration and judgment of the prescribing physician should be taken into account in deciding the proper amount. The determination of a therapeutically effective amount of a conjugate is well within the capabilities of one with skill in the art.

Although a therapeutically effective amount of a conjugate will vary according to the patient being treated, suitable doses will typically include between about 0.1 mg and 1000 mg of the compound. Preferably, a dose contains between about 0.1 mg and 500 mg of the compound. More preferably, a dose contains between about 0.1 mg and 250 mg of the compound.

In some cases, it may be necessary to use dosages outside of the stated ranges to treat a patient. Those cases will be apparent to the prescribing physician. Where it is necessary, a physician will also know how and when to interrupt, adjust or terminate treatment in conjunction with a response of a particular patient.

Conjugates may be administered in a single daily dose, or the total daily dosage may be administered in divided doses of two, three or four times daily. Tmax and Cmax for a conjugate in a subject (and the corresponding therapeutic agent not conjugated to an aminoadamantane) can be calculated using methods known in the art (see, e.g., the USP (United States Pharmacopoeia) and U.S. Pat. No.6,555,581). Tmax and Cmax can be calculated using samples extracted from brain or cerebrospinal fluid. Values can also be calculated based on samples taken from tissues such as serum. It is expected that altered serum values of a conjugate as compared to the therapeutic agent delivered when not conjugated to the therapeutic agent will reflect delivery of the conjugate from tissues outside the blood-brain barrier to the brain.

Combinations Containing Conjugates

The conjugate can be administered with another neuroprotectant, including a second conjugate as described herein, an anti-inflammatory agent, an immunosuppressant, an antiviral agent conjugate of the invention in combination with other conjugate of the invention.

Combination therapy” (or “co-therapy”) includes the administration of a conjugate of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents.

Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks. The conjugate and the other pharmacologically active agent may be administered to a patient simultaneously, sequentially or in combination. It will be appreciated that when using a combination of the invention, the compound of the invention and the other pharmacologically active agent may be in the same pharmaceutically acceptable carrier and therefore administered simultaneously. They may be in separate pharmaceutical carriers such as conventional oral dosage forms which are taken simultaneously. The term “combination” further refers to the case where the compounds are provided in separate dosage forms and are administered sequentially.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of targeting a therapeutic agent to the brain, the method comprising
administering to a subject in need thereof a conjugate comprising an aminoadamantane derivative linked to said therapeutic agent.

2. The method of claim 1, wherein said method further includes identifying a therapeutic agent in which targeting to the brain of a subject is desired.

3. The method of claim 1, wherein said therapeutic agent reaches a Tmax at from between about 0.5 hours to about 8 hours after administering to said subject.

4. The method of claim 1, reaches a Tmax at from about 1.0 hours to about 6 hours.

5. The method of claim 1, reaches a Tmax at from about 1.5 hours to about 2.5 hours.

6. The method of claim 1, wherein the Tmax of said therapeutic agent occurs in a time that is at least 1.5 times sooner than that of the Tmax that occurs when said therapeutic agent administered when not conjugated to said aminoadamantane derivative.

7. The method of claim 1, wherein the Tmax of said therapeutic agent occurs in a time that is at least 2.5 times sooner than that of the Tmax that occurs when said therapeutic agent administered when not conjugated to said aminoadamantane derivative.

8. The method of claim 1, wherein the Tmax of said therapeutic agent occurs in a time that is at least 4.0 times sooner than that of the Tmax that occurs when said therapeutic agent administered when not conjugated to said aminoadamantane derivative.

9. The method of claim 1, wherein said conjugate reaches Cmax in about 12 hours after administration of the product.

10. The method of claim 1, wherein said conjugate reaches Cmax in about 6 hours after administration of the product.

11. The method of claim 1, wherein said conjugate reaches Cmax about 1 hour after administration of the product.

12. The method of claim 1, wherein said conjugate reaches a Cmax in a time that is at least 1.5 times sooner than that of the Cmax that occurs when said therapeutic agent administered when not conjugated to said aminoadamantane derivative.

13. The method of claim 1, wherein said conjugate reaches a Cmax in serum in a time that is at least 2 times sooner than that of the Cmax that occurs when said therapeutic agent administered when not conjugated to said aminoadamantane derivative.

14. The method of claim 1, wherein said conjugate reaches a Cmax in a time that is at least 4 times sooner than that of the Cmax that occurs when said therapeutic agent administered when not conjugated to said aminoadamantane derivative.

15. The method of claim 1, wherein said therapeutic agent is a neuroprotective agent.

16. The method of claim 1, wherein said therapeutic agent is non-nitrosylated or is a non NO-generating therapeutic agent.

17. The method of claim 1, therapeutic agent is for treating a therapeutic agent is for treating a disorder associated with excessive NMDAR activity.

18. The method of claim 1, wherein said therapeutic agent is for treating a neurological disorder.

19. The method of claim 1, wherein said neurological disorder is selected from the group consisting of stroke, Alzheimer's disease, Parkinson's disease, epilepsy, metabolic disorders, glaucoma, HIV-associated dementia, neuropathic pain, Huntington's disease, anxiety, depression, and withdrawal from drug addiction or drug dependency.

20. The method of claim 1, wherein said therapeutic agent is for treating hyperhomocysteinemia contributing to atheroslcerotic and other degenerative disease processes.

21. The method of claim 1, wherein said therapeutic agent is for treating head trauma or spinal cord injury.

22. The method of claim 1, wherein said therapeutic agent is for treating a demyelinating disease.

23. The method of claim 22, wherein said demyelinating disease is multiple sclerosis.

24. The method of claim 2, wherein said therapeutic agent is for treating a disorder associated with excessive NMDAR activity.

25. The method of claim 1, wherein said therapeutic agent is selected from the group consisting of a caspase inhibitor, a superoxide dismutase mimetic, calcium chelator, MAPK antagonist, an ERK-MAPK antagonist, a Cytochrome C chelating antibody, APAF-1 inhibitor, AIF inhibitor/Apoptosis inhibitor, Gamma Vinyl GABA, PARP inhibitor, and an NOS inhibitor.

26. The method of claim 1, wherein said aminoadamantane derivative is neuroprotective.

27. The method of claim 1, wherein said aminoadamantane derivative binds to an N-methyl-D-aspartate (NMDA) receptor expressed on an injured neuron.

28. The method of claim 1, wherein said aminoadamantane derivative is memantine.

29. The method of claim 1, wherein said aminoadamantane derivative is rimantadine (1-(1-aminoethyl)adamantane).

30. The method of claim 1, wherein said aminoadamantane derivative is amantadine (1-aminoadamantane)

31. The method of claim 1, wherein said therapeutic agent is attached to the aminoadamantane derivative in said conjugate at the 1-(bridgehead) position or the 2-position.

32. The method of claim 1, wherein said therapeutic agent is attached to the aminoadamantane derivative via an unstable linkage.

33. A method of targeting a therapeutic agent to an injured neuron of a subject, the method comprising administering to a subject in need thereof a conjugate comprising an aminoadamantane derivative linked to said therapeutic agent.

34. The method of claim 33, wherein the injured neuron is in the brain of said subject.

35. A method of lowering the neurotoxicity of a therapeutic agent in a subject, the method comprising administering to said subject a conjugate comprising an aminoadamantane derivative linked to said therapeutic agent.
